(19) United States
(12) Patent Application Publication
(10) Pub. No.: US 2005/0226434 A1
(43) Pub. Date: Oct. 13, 2005

(54) Noise reduction systems and methods

(76) Inventors: John P. Franz, Houston, TX (US); Reza M. Bacchus, Spring, TX (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/816,695

(22) Filed: Apr. 1, 2004

(51) Int. Cl. A61F 11/06; G10K 11/16; H03B 29/00

(52) U.S. Cl. 381/71.7; 381/71.1; 381/94.1

(57) ABSTRACT

Noise reduction methods and systems are described. In one embodiment, a user location system is configured to determine a user's location within a room. A noise profile processing unit is configured to produce a remedial noise profile specific to the user's location for use in reducing user-perceivable noise at the user's location.

### BACKGROUND

Various types of rooms can have various types of noise that can, from time to time, be distracting or annoying for individuals who work in or otherwise find themselves in such rooms. As an example, consider the following.

Servers are types of computers that can perform different types of functions. For example, web servers typically can provide access to the Internet and data servers can store and provide data to users. It has become a common practice in various industries that utilize servers to group multiple servers together in the same room or in a common facility. The reason for this is that for large scale operations, multiple servers are generally needed. For example, for a company that provides a large scale Internet-based email system, the company's servers might number in the hundreds and be located in a central facility so that managers, administrators and service personnel can be located at a single location.

As server technology continues to improve, the density of servers continues to grow larger. For example, the computer industry is producing smaller and smaller components such as hard drives, power supplies and the like. As the size of these components is reduced, individual servers and collections of servers become denser.

One of the impacts of increased server and server collection densities pertains to cooling the servers. Specifically, as servers grow denser and denser, there is a natural tendency for the servers to produce more and more heat. As a result, cooling fans have been used in the past to maintain server temperatures within an acceptable range. As the densities of the servers have increased, fan technology has had to adapt to these increases. Specifically, with denser, smaller servers, smaller fans that operate at very high speeds (e.g. 8000, 10,000 and 14,000 RPM fans) have been developed. One of the downsides of these smaller higher-RPM fans is the noise that the fans produce. The noise problem is even more compounded when multiple servers are placed into server racks, and multiple server racks are placed in large rooms—often referred to as data centers. Thus, one of the noise problems associated with these higher density servers is associated with the additive effect of multiple servers or server racks.

In addition to this additive effect, there can also be a noise problem that can emanate from computer room air conditioning (CRAC) systems that are utilized in large rooms where multiple servers or server racks are employed. Specifically, large collections of servers can require a tremendous amount of cooling from the CRAC system. In some arrangements, CRAC cooling is performed by using an under-the-floor system in which are ducts under the floor vent cool air through tiles in the floor and take warmer air in from the ceiling for recycling. Needless to say, the CRAC systems can also add to the noise in the environment in which the servers are located.

### BRIEF DESCRIPTION OF THE DRAWINGS

FIG. 1 is an isometric view of an exemplary system in accordance with one embodiment.

FIG. 2 is a block diagram of exemplary components of a system in accordance with one embodiment.

FIG. 3 is a block diagram of a noise profile processing unit in accordance with one embodiment.

FIG. 3a is a block diagram of a noise profile processing unit in accordance with one embodiment.

FIG. 4 is a flow diagram that describes a method in accordance with one embodiment.

FIGS. 5 and 6 are isometric views similar to FIG. 1 and are utilized to describe an illustrative example in accordance with one embodiment.

### DETAILED DESCRIPTION

Overview

In various embodiments described in this document, a noise reduction system is employed in an environment to reduce the noise that a user within that environment experiences. In at least some embodiments, the system determines the user's location within the environment. The environment can comprise any suitable environment an example of which is a server environment comprising a room in which multiple servers are located. The system also ascertains a noise profile associated with the user's location. Once the user's location and associated noise profile are ascertained, the system processes the noise profile to produce a remedial noise profile that is configured to reduce the noise that the user experiences at their current location. A signal that represents the remedial noise profile is then used to drive one or more speakers proximate the user's location so that the user experiences less noise at their location.

Exemplary Embodiment

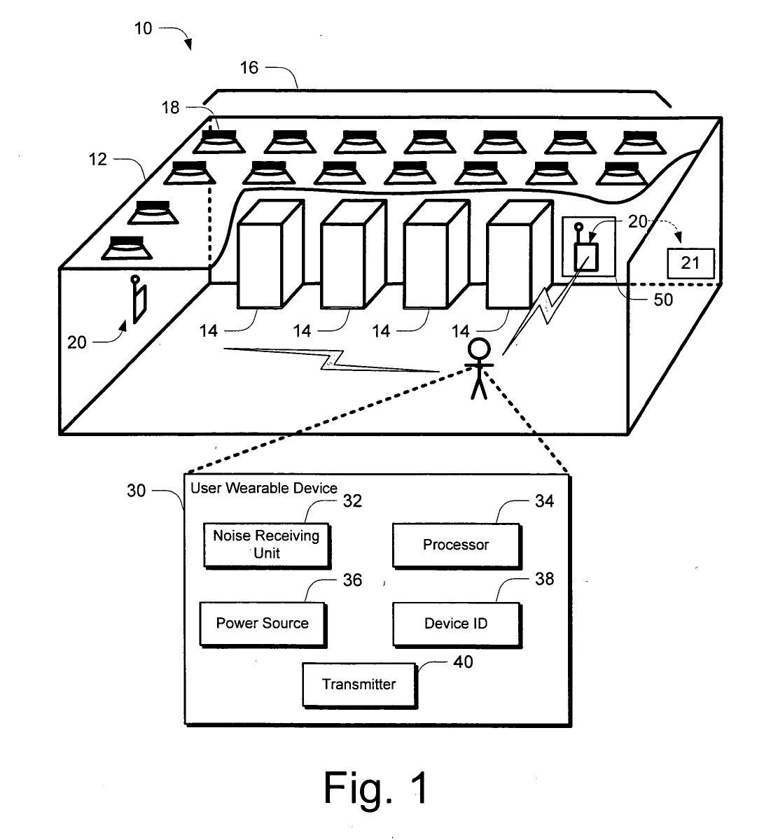

FIG. 1 shows an exemplary system in accordance with one embodiment generally at 10. In this example, system 10 comprises a room 12 in which multiple server racks 14 are located. Each server rack typically contains multiple servers as will be appreciated by the skilled artisan.

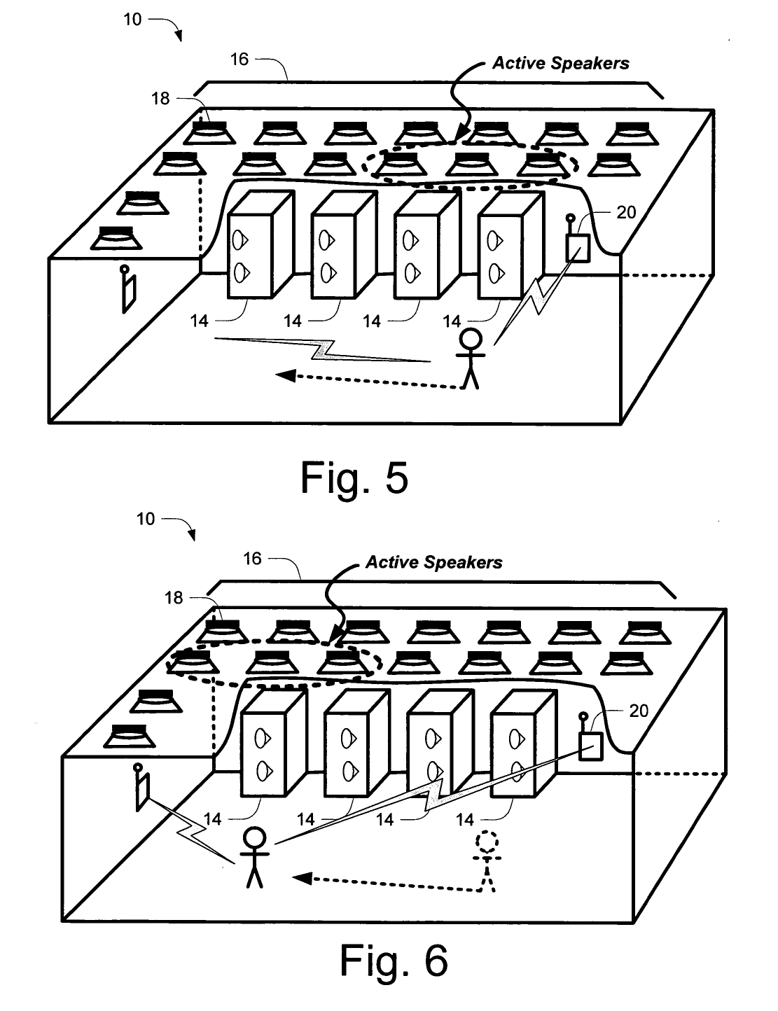

In accordance with the described embodiment, room 12 comprises a speaker array 16 which includes one or more speakers 18. In the present example, the speakers are mounted in the ceiling. It is to be appreciated and understood that the speakers of the speaker array could be mounted anywhere in or around the room. One example of alternate speaker locations is provided below in connection with the example of FIGS. 5 and 6.

A user location system 20 is provided and is configured to locate or facilitate locating a user within the room. In the present example, the user location system 20 comprises multiple wall mounted units, two of which are shown, that are utilized to triangulate or otherwise ascertain the user's position within the room, as will become apparent below. It is to be appreciated that any suitable means can be utilized to ascertain the user's location such as motion detection, body heat sensors, and the like.

The user location system comprises part of a noise reduction unit 50 that includes components that are configured to reduce the noise that a user experiences, as will become apparent below. Although noise reduction unit 50 is shown to contain user location system 20, such is simply intended to represent that the user location system 20 comprises a constituent part of an overall noise reduction unit. As such, other components of the noise reduction unit 50 can reside at other locations within or outside of the room of interest.

In the described embodiment, a user is provided with a user-wearable device 30 which can take any suitable form such as a wearable badge and the like. In accordance with one embodiment, device 30 comprises a noise-receiving unit 32, a processor 34, a power source 36, a unique device ID 38 and a transmitter 40.

In one embodiment, noise-receiving unit 32 comprises a microphone or other suitable transducer that is configured, under the influence of power source 36, to pick up noise in and around the user's location. Processor 34 is coupled with the noise-receiving unit and processes the noise signal received by the noise-receiving unit to ascertain a noise profile that is associated with the user's current location. Transmitter 40 is coupled with the processor and is configured to transmit data associated with the noise profile to an off-device location, such as noise reduction unit 50, for further processing as described below. Transmitter 40 can also transmit the device ID 38 with or separately from the noise profile data so that the receiving system can identify which badge, and hence which user, is associated with which noise profile data. Although only one user is shown, it is to be understood that the system is capable of handling multiple users who might be in the room.

As noted above, system 10 is configured to ascertain the user's location. In one embodiment, user location system 20 receives transmissions from device 30 and is able to triangulate or otherwise ascertain the user's position within the room. Techniques for triangulation are known and will be understood by the skilled artisan. Accordingly, for the sake of brevity, such techniques are not described in great detail herein. In short though, triangulation systems, in general, utilize trigonometric methods to determine the position of a location from the angles to it, or the time that it takes for a transmitted signal to reach a receiver at two or more fixed, known locations. In the present example, the user location systems 20 transmit a signal which the user wearable device 30 receives and retransmits. In this case, the difference in arrival times of the re-transmissions from the device 30 at each of the user location systems 20 can be analyzed and used to determine the user's location. This analysis can be conducted by the individual systems 20, or by other components in association with systems 20.

For example, in some embodiments, each user location system 20 comprises a beacon. A master unit 21 can instruct each beacon to perform a frequency sweep throughout the room. If a user-wearable device is in the room and is interrogated by the beacons, the device can then send a response, including its ID, to each of the individual systems 20. Each system can then perform an analysis on the transmission and receive times between the signals transmitted from the respective beacon and the signals received back from each of the user-wearable devices. Each system 20 can then send this information to master unit 21 which can then compare and analyze all of the beacons' information to ascertain the locations of associated user-wearable devices. Once the locations of all of the user-wearable devices are ascertained, in this embodiment, the master unit 21 can send a message to each device for the device to send its associated noise profile data for processing as described below.

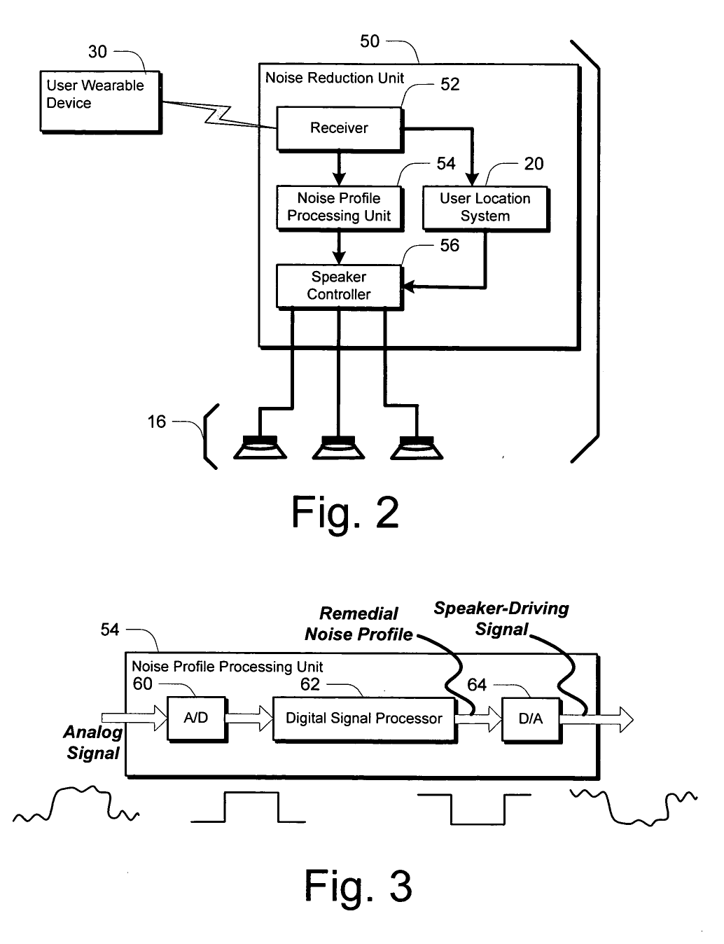

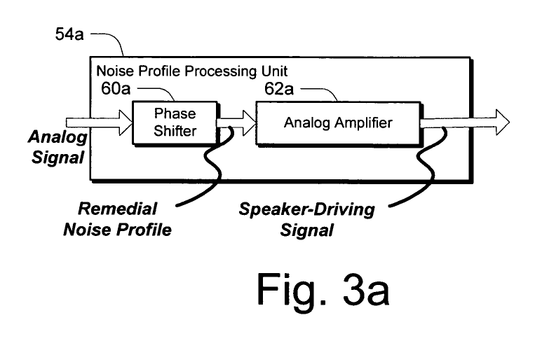

FIGS. 2 and 3 show additional components of system 10 in accordance with one embodiment. In this example, the user wearable device 30 is configured to communicate with noise reduction unit 50. Noise reduction unit 50 may or may not comprise master unit 21. In one embodiment, unit 50 comprises a receiver 52, a noise profile processing unit 54, a speaker controller 56 and, in at least some embodiments, user location systems 20. In this example, user location system 20 constitutes the same user location system that is illustrated in FIG. 1. It is to be appreciated and understood that in other embodiments, user wearable device 30 can transmit to a user location system 20 and, in turn, user location system 20 can provide the appropriate data for analysis to a noise reduction system that includes components 52, 54 and 56.

In operation, receiver 52 receives transmissions from the user wearable device 30. The transmissions include data associated with the noise profile that is picked up by the user's device at their particular location, and can also include the device ID. The transmitted data can comprise any suitable data that can be processed as described below. For example, the noise profile data can take the form of an analog signal that is transmitted from the device 30 to the unit 50. Alternately, the data can take the form of a digitized version of the analog noise signal that is sensed by the device 30.

Receiver 52 is coupled with a noise profile processing unit 54 which is configured to receive the noise profile data and process the noise profile data to produce a remedial noise profile that can reduce the amount of noise that the user experiences. In developing the remedial noise profile, standard noise reduction techniques can be utilized, as will be appreciated by the skilled artisan.

For example, consider FIG. 3 which illustrates components of an exemplary noise profile processing unit 54 in accordance with one embodiment. In this example, noise profile processing unit 54 comprises an A/D converter 60, a digital signal processor 62, and a D/A converter 64. In operation, in this particular example, an analog signal is received by the noise profile processing unit 54 and digitized via A/D converter 60. The digitized version of the signal is then provided to a suitably configured digital processor 62 which processes the signal to produce a remedial noise profile. A suitable remedial noise profile can comprise, for example, a signal that is inverted relative to the signal received by the digital signal processor. The remedial noise profile is then provided to D/A converter 64 which produces an analog speaker-driving signal that is used to drive the appropriate speaker(s).

Alternately or additionally, consider FIG. 3a which illustrates a noise profile processing unit 54a in accordance with another embodiment. Here, unit 54a comprises a phase shifter 60a configured to phase shift an analog signal, and amplifier 62a to amplify the phase shifted signal which is then used to drive the appropriate speaker(s).

The principles of such noise cancellation systems are known by the skilled artisan and, for the sake of brevity, are not described in additional detail here. For example, for a treatment of exemplary noise cancellation systems in various environments, the reader is referred to the following U.S. Pat. Nos. 6,445,799; 6,654,467; 6,278,786; and 6,232,994.

Referring back to FIG. 2, speaker controller 56 is operably connected to speakers of speaker array 16 and is used to drive one or more of the speakers. The speaker controller 56 can drive the speakers wirelessly, and/or through a wired connection. Using the location information ascertained from user location system 20, as well as the output of the noise profile processing unit 54, the speaker controller 56 can drive the appropriate speakers proximate an associated user with a signal embodying the remedial noise profile such that the user-perceived noise at their particular location is reduced.

In the illustrated and described embodiment, the user-wearable device 30, the user location system 20, and the noise reduction unit 50 monitor the user's location within the room, as well as the noise profile experienced by the user so that if the user's location and/or experienced noise profile changes sufficient to require a new remedial noise profile, the noise reduction unit 50 can produce an appropriate remedial noise profile.

Exemplary Method

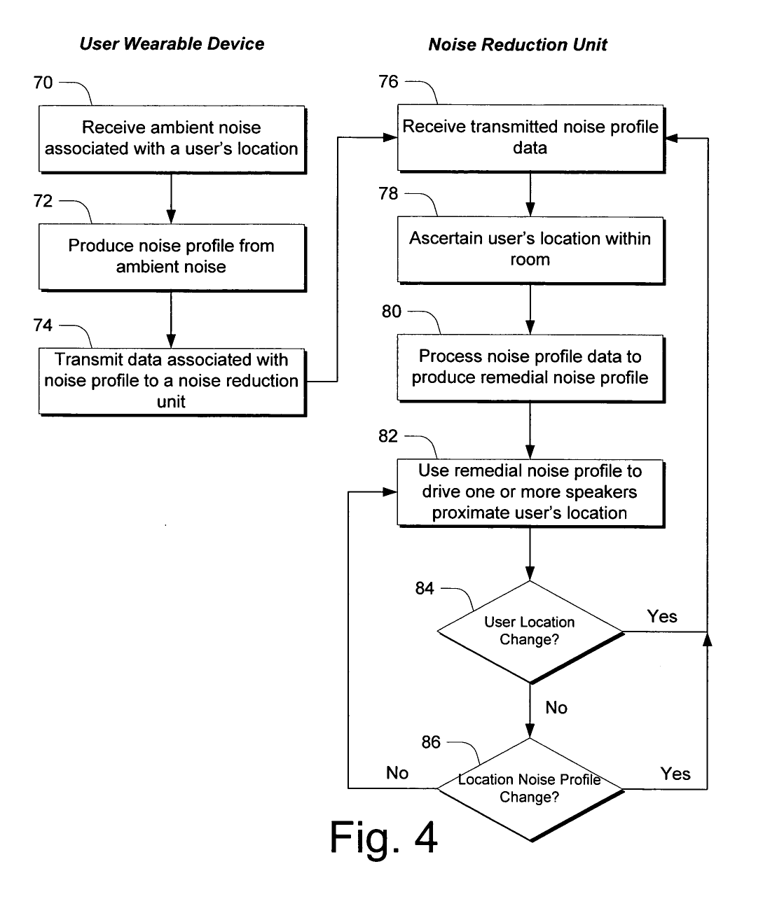

FIG. 4 illustrates a method in accordance with one embodiment. The method can be implemented using any suitable hardware, software, firmware or combination thereof. In one embodiment, the method can be implemented utilizing a system such as the one shown and described above in FIGS. 1-3. As such, in the present discussion, the method of FIG. 4 is described in the context of acts that are or can be performed by the user wearable device, and acts that are or can be performed by the noise reduction unit.

Step 70 receives ambient noise associated with a user's location. In one embodiment, the user's location is within a room in which multiple servers reside. This step can be performed in any suitable manner. For example, in some embodiments, the user can wear a device, such as a badge, that includes componentry that is configured to receive the ambient noise. One example of a user wearable device is provided above in FIG. 1. Step 72 produces a noise profile from the ambient noise. This noise profile can comprise an analog signal that is produced by the on-device componentry. Alternately, the noise profile can comprise a digitized version of the analog signal. Step 74 transmits data associated with the noise profile to a noise reduction unit, such as the one shown and described at 50 in FIG. 2. This step can also transmit a device ID that uniquely identifies the user wearable device. Transmission of the device ID can occur together with transmission of the noise profile data. Alternately, the device ID can be transmitted separately.

Step 76 receives transmitted noise profile data and step 78 ascertains the user's location within the room. Step 78 can be performed in any suitable way and need not be performed in the order implied in the flow diagram. In but one embodiment, a signal that is transmitted from the user's wearable device is used to triangulate the user's position within the room. In this example, the transmitted signal is used to both convey the noise profile to the noise reduction unit and to ascertain the user's location within the room. It is to be appreciated, however, that any suitable means can be used to ascertain the user's location within the room. By ascertaining the user's location in the room, the system can ascertain which of one or more speakers of a speaker array to drive to reduce the noise to which the user is exposed.

Step 80 processes the noise profile data to produce a remedial noise profile. The remedial noise profile is desirably one that can be used to reduce the noise to which the user is exposed in the room. Step 82 uses the remedial noise profile in driving one or more speakers proximate the user's location. In one embodiment, the remedial noise profile can be utilized to produce a driving signal that is used to drive selected speakers proximate the user's location.

Steps 84 and 86 provide a feedback loop that is designed to mitigate noise effects as a user's environmental conditions change. Specifically, step 84 ascertains whether the user's location has changed. This step can be performed, in at least some embodiments, by periodically or continuously triangulating and re-triangulating the user's location. Where other means are utilized to ascertain the user's location, those means can be employed periodically or continuously. If the user's location changes, then the method can return to step 76 which receives transmitted noise profile data at the new location and repeat steps 78 and on.

If, on the other hand, the user's location does not change, step 86 ascertains whether the location noise profile has changed. For example, if the user remains in the same location but for some reason the ambient noise at that location meaningfully changes (e.g. if the cooling fans speed up or slow down), then the method returns to step 76 as described above. If the location noise profile does not change, then the method returns to step 82 which uses the same remedial noise profile as was first produced to continue to drive the speaker(s) proximate the user's location. It should be appreciated and understood that in at least some embodiments, the noise profile that is the subject of the processing that takes place in step 86 can be the original noise profile that caused the remedial noise profile to be produced. This original noise profile can be, for example, stored in memory on the noise reduction unit. As the remedial noise profile can effectively cause a reduction in the ambient noise experienced by the user such that the resultant noise profile would be dramatically reduced, the system can monitor for a change in the profile that originally caused the remedial noise profile to be produced. In this way, the system can be considered as a continuous feedback system. It is also to be appreciated and understood that while the acts performed at steps 84 and 86 are shown in a serial order, such acts can be independently performed, for example, in parallel.

It is to be appreciated and understood that threshold conditions can be set up and used to determine whether to produce a new remedial noise profile. For example, it is possible in some circumstances for a user to move around the room, and yet the ambient noise to which the user is exposed remains the same. In this situation, the system can utilize a comparative analysis to ascertain whether the received transmitted data (step 76) responsive to the user's movements varies in a defined manner from previously-received noise profile data. If not, then the previously-used remedial noise profile might then continue to be used to drive a different collection of speakers until the newly-received transmitted data exceeds some threshold condition.

In this way, the above described system is adaptive in that as a user moves and/or the noise profile at the user's present location changes, a remedial noise profile is produced and used to reduce the ambient noise that the user experiences.

In at least some embodiments, one means that the noise reduction unit can utilize for producing the remedial noise profile is a PID (Proportional, Integral, Derivative) Loop. PID loops and their operation will be generally understood by the skilled artisan. In brief summary though, a PID Loop measures the output of a process and calculates the difference or error between what is measured and what is referred to as a set point. If there is a difference or error, the PID Loop or an associated controller can adjust its output to alter the process to bring it closer to the desired set point, thus reducing or minimizing the error. In the present example, the set point can be established as the zero noise or low noise condition sensed at the location of the user/badge.

Illustrative Example

As an illustrative example, consider FIGS. 5 and 6. In FIG. 5, a user is positioned on the right side of the room. As a result of the processing described above, three speakers (encircled by the dashed oval) of speaker array 16 are active and being driven by a signal that embodies the remedial noise profile for the associated location. In at least some embodiments, one or more speakers can be mounted in or on a server rack door. For example, each of the illustrated server racks 14 is shown to have a pair of speakers (not specifically designated). In these particular embodiments, one or more of the server rack door speakers can be driven by a signal that embodies the remedial noise profile. In this particular example, the speakers of the rightmost server rack 14 might be driven by such a signal. Consider now that the user moves to a new position as shown in FIG. 6. In this case, the user's new location is ascertained and a new remedial noise profile, if necessary, is produced and utilized to drive a new collection of active speakers (encircled by the dashed oval). In embodiments that include server rack door speakers, a new collection of server rack door speakers might be driven by a signal that embodies the new remedial noise profile. In the present example, the speakers of the leftmost server rack 14 might be driven by such a signal.

Conclusion

The above-described systems and methods provide an adaptive approach to reduce the noise that users experience in various types of rooms in which the users may find themselves. In embodiments where such rooms constitute those in which multiple servers or computers reside, by providing solutions that are adaptive to changing noise profiles and/or user locations, it is possible to use more cooling fans and even louder cooling fans to extend air cooling of server and computer systems.

Although the invention has been described in language specific to structural features and/or methodological steps, it is to be understood that the invention defined in the appended claims is not necessarily limited to the specific features or steps described. Rather, the specific features and steps are disclosed as preferred forms of implementing the claimed invention.

1. A system comprising:
a user location system configured to determine a user's location within a room; and
a noise profile processing unit configured to produce a remedial noise profile specific to the user's location for use in reducing user-perceivable noise at the user's location.

2. The system of claim 1, wherein the user location system is configured to determine a user's location by triangulating the user's position within the room.

3. The system of claim 1 further comprising a receiver configured to receive transmitted data associated with a noise profile associated with the user's location, wherein the noise profile processing unit is configured to produce the remedial noise profile from the transmitted noise profile.

4. The system of claim 1 further comprising a receiver configured to receive transmitted data associated with a noise profile associated with the user's location, wherein the noise profile processing unit is configured to produce the remedial noise profile from the transmitted noise profile, wherein the receiver is configured to receive the transmitted data from a user wearable device.

5. The system of claim 1 further comprising a speaker controller for controlling a plurality of speakers operably associated with the room, and wherein the system is configured to select and drive, via the speaker controller, one or more speakers proximate the user's location.

6. The system of claim 1, wherein the system is configured to produce a remedial noise profile responsive to a change in one or more of the user's location and the ambient sound associated with the user's location.

7. The system of claim 1, wherein the system is configured to use a previously used remedial noise profile responsive to change in the user's location.

8. A system comprising:
a room;
a speaker array comprising multiple speakers positioned around the room;
a user location system configured to determine a user's location within the room;
a noise profile processing unit configured to produce a remedial noise profile from data transmitted from one or more user-wearable devices, said transmitted data being associated with a noise profile that is associated with the user's location, said remedial noise profile being specific to the user's location and configured for use in reducing user-perceivable noise at the user's location; and
a speaker controller for controlling speakers of the speaker array, the speaker controller being configured to select speakers proximate the user's location and drive the selected speakers with a signal that embodies the remedial noise profile.

9. The system of claim 8, wherein at least some of the speakers are mounted in a room ceiling.

10. The system of claim 8, wherein at least some of the speakers are mounted on at least one server rack within the room.

11. The system of claim 8, wherein the user location system comprises means for triangulating the user's location.

12. The system of claim 8 further comprising one or more user-wearable devices each configured to transmit data associated with a noise profile associated with the user's location, wherein said one or more user-wearable devices comprise a noise receiving unit for receiving ambient noise associated with the user's location and a processor for producing, from the ambient noise, said noise profile data.

13. The system of claim 8 further comprising one or more user-wearable devices each configured to transmit data associated with a noise profile associated with the user's location, wherein said one or more user-wearable devices comprise a noise receiving unit for receiving ambient noise associated with the user's location and a processor for producing, from the ambient noise, said noise profile data, wherein said one or more user-wearable devices each comprise a unique ID, each device being configured to transmit its unique ID with its associated noise profile data.

14. The system of claim 8, wherein the system is configured to produce a remedial noise profile responsive to change in one or more of the user's location and the noise profile associated with the user's location.

15. The system of claim 8, wherein the noise profile processing unit comprises at least:
an A/D converter for receiving an analog signal and producing a corresponding digital signal;
a digital signal processor for receiving the corresponding digital signal and producing a remedial noise profile; and
a D/A converter for receiving the remedial noise profile and producing the signal that embodies the remedial noise profile.

16. The system of claim 8, wherein the noise profile processing unit comprises at least:
a phase shifter configured to phase shift an analog signal to provide a remedial noise profile; and
an amplifier connected with the phase shifter and configured to amplify the remedial noise profile for driving one or more speakers of the speaker array.

17. A system comprising:
a user wearable device comprising:
a noise receiving unit to receive ambient noise associated with a user's location within a room;
a processor configured to process the received ambient noise to ascertain a noise profile associated with the user's location;
a device ID that uniquely identifies the device; and
a transmitter configured to transmit both data associated with the noise profile and the device ID to an off-device location so that the noise profile and device ID can be utilized to ascertain the user's location and produce a remedial noise profile that can be used in reducing user-perceivable noise at the user's location.

18. The system of claim 17, wherein the user-wearable device comprises a badge.

19. A method comprising:
receiving ambient noise associated with a user's location within a room in which multiple servers reside;
producing a noise profile from the ambient noise;
transmitting data associated with the noise profile to a noise reduction unit; and
transmitting an ID that uniquely identifies the user for location determination.

20. The method of claim 19, wherein said act of receiving is performed by a user-wearable device.

21. The method of claim 19, wherein said act of receiving is performed by a user-wearable device, and said act of transmitting an ID comprises transmitting a device ID that uniquely identifies the user-wearable device.

22. A method comprising:
receiving transmitted data associated with a noise profile of ambient noise associated with a user's location within a room;
ascertaining the user's location within the room;
responsive to ascertaining the user's location within the room, selecting one or more speakers proximate the user's location;
processing the noise profile data to produce a remedial noise profile that can be used to reduce the noise to which the user is exposed in the room; and
using the remedial noise profile in driving said selected one or more speakers.

23. The method of claim 22, wherein said act of ascertaining the user's location is performed by triangulating the user's location using a signal transmitted from a user-wearable device.

24. The method of claim 22, wherein said act of ascertaining the user's location is performed by triangulating the user's location using a signal transmitted from a user-wearable device, and wherein the signal that is transmitted comprises the noise profile data.

25. The method of claim 22 further comprising ascertaining whether the user's location changes and if so:
receiving transmitted data associated with a noise profile at a new location;
ascertaining the user's new location;
responsive to ascertaining the user's new location within the room, selecting one or more speakers proximate the user's new location;
processing the noise profile data associated with the new location to produce a new remedial noise profile that can be used to reduce the noise to which the user is exposed in the room; and
using the new remedial noise profile in driving said selected one or more speakers proximate the user's new location.

26. The method of claim 22 further comprising ascertaining whether the user's location changes and if so:
receiving transmitted data associated with a noise profile at a new location; ascertaining the user's new location;
responsive to ascertaining the user's new location within the room, selecting one or more speakers proximate the user's new location;
processing the noise profile data associated with the new location to ascertain whether a new remedial noise profile is needed for the new location;
if a new remedial noise profile is needed for the new location:
processing the noise profile data associated with the new location to. produce a new remedial noise profile that can be used to reduce the noise to which the user is exposed in the room at the new location; and
using the new remedial noise profile in driving said selected one or more speakers proximate the user's new location;
otherwise, using the first-mentioned remedial noise profile in driving said one or more speakers proximate the user's new location.

27. The method of claim 22 further comprising ascertaining whether the user's location noise profile changes and if so:
receiving transmitted data associated with a new noise profile;
processing the new noise profile data to produce a new remedial noise profile that can be used to reduce the noise to which the user is exposed in the room; and
using the new remedial noise profile in driving said selected one or more speakers.

28. The method of claim 22 further comprising responsive to both the user's location changing and the location noise profile changing:
receiving transmitted data associated with a noise profile at a new location;
ascertaining the user's new location;
responsive to ascertaining the user's new location within the room, selecting one or more speakers proximate the user's new location;
processing the noise profile data associated with the new location to produce a new remedial noise profile that can be used to reduce the noise to which the user is exposed in the room; and
using the new remedial noise profile in driving said selected one or more speakers.

29. A system comprising a user wearable device and a noise reduction unit associated with a room in which multiple servers are located, the user wearable device and the noise reduction unit cooperating to facilitate an adaptive reduction in ambient noise proximate the user wearable device as the device is moved from location to location within the room.

30. A system comprising:
means for receiving transmitted data associated with a noise profile of ambient noise associated with a user's location within a room in which multiple servers are located;
means for ascertaining the user's location within the room;
means, responsive to said means for ascertaining the user's location within the room, for selecting one or more drivable speakers proximate the user's location;
means for processing the noise profile data to produce a remedial noise profile that can be used to reduce the noise to which the user is exposed in the room; and
means for using the remedial noise profile in driving said selected one or more speakers.
